(19) United States
(12) Patent Application Publication
(10) Pub. No.: US 2020/0359640 A1
(43) Pub. Date: Nov. 19, 2020

(54) METHODS FOR PRODUCING FERMENTED MILK AND LACTIC ACID BACTERIA STARTER

(71) Applicant: MEIJI CO., LTD., Tokyo (JP)

(72) Inventors: Hiroshi HORIUCHI, Kanagawa (JP); Yoshitaka KAWAI, Kanagawa (JP); Nao TAKAGI, Kanagawa (JP)

(73) Assignee: MEIJI CO., LTD., Tokyo (JP)

(21) Appl. No.: 16/959,781

(22) PCT Filed: Jan. 4, 2019
(86) PCT No.: PCT/JP2019/000017

§ 371 Date: Jul. 2, 2020

(30) Foreign Application Priority Data
Jan. 5, 2018 (JP) 2018-000755

(51) Int. Cl. A23C 9/123; C12N 1/20

(52) U.S. Cl. CPC A23C 9/123; C12N 1/20; A23Y 2220/37; A23Y 2240/75; A23Y 2220/15

(57) ABSTRACT

This method for producing fermented milk comprises: a starter inoculation step for inoculating a raw material milk with a starter to obtain a fermented milk base material; a fermentation step for fermenting the fermented milk base material to obtain a fermented milk; and a lactic acid bacteria addition step for adding lactic acid bacteria to the fermented milk.

### TECHNICAL FIELD

The present invention relates to a method for producing fermented milk such as yogurt. Further, the present invention relates to a method for producing a lactic acid bacterium starter used for producing fermented milk.

### BACKGROUND ART

In recent years, a lactic acid bacterium having a positive effect on a human body (hereinafter referred to as a “probiotic lactic acid bacterium”) has been attracting attention. It is believed that the probiotic lactic acid bacterium contributes to prevention of a lifestyle-related disease or the like by improving and making healthier the inside of the gastrointestinal tract and immunoregulatory action associated therewith. As the probiotic lactic acid bacterium, Lactobacillus gasseri, Lactobacillus bulgaricus, Lactobacillus plantarum, Lactobacillus casei, Bifidobacterium, and the like are well known.

Further, fermented milk is known as food for ingesting the lactic acid bacterium. The fermented milk is obtained by fermenting milk or the like with a lactic acid bacterium or yeast and then preparing it into a paste or a liquid form. However, a low pH environment of the fermented milk after fermentation has been considered as not being suitable for survival of the lactic acid bacteria. In particular, in a fermented milk product including the probiotic lactic acid bacteria, a certain number or more of the probiotic lactic acid bacteria are required to survive for a predetermined period of time from production to the expiration date. However, the number of the lactic acid bacteria gradually decreases with the lapse of the storage days of the product.

Thus, as a means of improving the viability of the lactic acid bacteria in the fermented milk, a method of adding a viability improving agent to a raw material of the fermented milk is known (Patent Literature 1). Patent Literature 1 proposes a viability improving agent of the lactic acid bacteria including a propionic acid bacteria fermented product and a method for improving the viability of the lactic acid bacteria using the viability improving agent. In this method, after a yogurt raw material including milk, powdered skim milk, and sweetener is sterilized by heating, a starter, a probiotic lactic acid bacterium, and a propionic acid bacterium fermented product are simultaneously added to the yogurt raw material to perform fermentation until the lactic acid acidity reaches 0.75%, thereby producing yogurt (fermented milk).

### CITATION LIST

### Patent Literature

Patent Literature 1: JP 2014-97081 A

### SUMMARY OF INVENTION

### Technical Problem

As described above, it can be expected that the viability of the lactic acid bacteria in the fermented milk can be improved by using the viability improving agent. However, using the viability improving agent not only requires a specific treatment for adjusting the viability improving agent but also increases a raw material cost, causing a concern of increasing a cost in the production of the fermented milk. Further, when the propionic acid bacterium fermented product or the like is added to the raw material of the currently commercially available fermented milk product for improving the viability of the lactic acid bacteria in the fermented milk, a flavor or the like of the fermented milk product may change, likely causing confusion to consumers.

Thus, an object of the present invention is to improve the viability of the lactic acid bacteria without relying on the viability improving agent.

### Solution to Problem

As a result of intensive studies on a means of solving the abovementioned problems, the inventors of the present invention have found that, after raw material milk is inoculated with a starter and fermented, further addition of a lactic acid bacterium can improve the viability of the lactic acid bacterium of the fermented milk during storage. Then, the present inventors have realized that the problems of the prior art can be solved based on the above knowledge and thereby completed the present invention. Specifically, the present invention includes the following steps.

A first aspect of the present invention relates to a method for producing fermented milk. The method for producing fermented milk according to the present invention includes a starter inoculation step, a fermentation step, and a lactic acid bacterium addition step. In the starter inoculation step, a starter is inoculated to raw material milk to obtain a fermented milk base material. In the fermentation step, the fermented milk base material is fermented to obtain fermented milk. In the lactic acid bacterium addition step, a lactic acid bacterium is added to the fermented milk. The “lactic acid bacterium” described in this step may be the same as or different from a lactic acid bacterium included in the starter.

As described above, the inventors have succeeded in significantly improving the viability of the lactic acid bacteria during storage of the fermented milk by inoculating the starter to the raw material milk for fermentation and then further adding the lactic acid bacteria to the fermented raw material milk. Conventionally, all of the lactic acid bacteria are usually added to the raw material milk before fermentation. However, the viability of the lactic acid bacteria can be improved just by changing a timing of the addition of the lactic acid bacteria to later than the fermentation of the raw material milk. This makes it possible to improve the viability of the lactic acid bacteria in the fermented milk without relying on a viability improving agent. Further, since the viability of the lactic acid bacteria added after the fermentation tends to improve, it becomes easy to improve the viability of only specific lactic acid bacteria of interest. That is, the viability of the lactic acid bacteria (starter) added before the fermentation remains the same, while the viability of the lactic acid bacteria added after the fermentation improves. This makes it easy to control the viability of each type of lactic acid bacteria in the fermented milk. Note that adding a viability improving agent to the raw material milk is not excluded from the scope of the present invention. That is, not using a viability improving agent is not a prerequisite for the present invention.

In the method for producing the fermented milk according to the present invention, the lactic acid bacteria added to the fermented milk in the lactic acid bacterium addition step preferably include a probiotic lactic acid bacterium. Examples of the probiotic lactic acid bacterium include Lactobacillus gasseri, Lactobacillus bulgaricus, Lactobacillus plantarum, Lactobacillus casei, or Bifidobacterium.

In the present invention, the starter inoculated to the raw material milk in the starter inoculation step preferably includes both or either of Lactobacillus bulgaricus and Streptococcus thermophilus. In this case, as the lactic acid bacterium added to the fermented milk in the lactic acid bacterium addition step, the lactic acid bacterium different from the one included in the above starter may be adopted.

In the method for producing the fermented milk according to the present invention, the fermentation step is preferably a step in which a fermented milk base material is fermented until the acidity of the fermented milk base material reaches 0.7% or more. In this manner, the lactic acid bacteria are added after the fermentation of the fermented milk base material sufficiently progresses, so that the viability of the lactic acid bacteria can be more effectively improved.

The method for producing the fermented milk according to the present invention preferably further includes a stirring step. In the stirring step, the fermented milk is stirred after the fermentation step but before the lactic acid bacterium addition step. In this manner, the lactic acid bacteria are added after the fermented milk is stirred into a paste or a liquid form, so that the lactic acid bacteria are prevented from being damaged by the stirring treatment, making it possible to improve the viability of the lactic acid bacteria more effectively.

A second aspect of the present invention relates to a method for producing a lactic acid bacterium starter. The lactic acid bacterium starter is used for obtaining the fermented milk by fermenting the raw material milk. The method for producing the lactic acid bacterium starter includes a starter inoculation step, a fermentation step, and a lactic acid bacterium addition step. In the starter inoculation step, the starter is inoculated to a medium including a milk component. In the fermentation step, the medium after the starter inoculation step is fermented. In the lactic acid bacterium addition step, the lactic acid bacteria are added to the medium after the fermentation step. Producing the lactic acid bacterium starter in this manner can improve the viability of the lactic acid bacteria during storage period.

### Advantageous Effects of Invention

According to the present invention, the viability of the lactic acid bacteria can be improved without relying on the viability improving agent. In particular, the number of the lactic acid bacteria gradually decreases with the lapse of the storage days of the fermented milk product. However, improving the viability of the lactic acid bacteria can stabilize the quality of the fermented milk product and extend the expiration date thereof. Further, improving the viability of the lactic acid bacteria can reduce an addition amount of the lactic acid bacteria and thus reduce a raw material cost.

### BRIEF DESCRIPTION OF DRAWINGS

FIG. 1 is a flowchart illustrating each step of a method for producing fermented milk according to the present invention.

FIG. 2 is a flowchart illustrating each step of a method for producing a lactic acid bacterium starter according to the present invention.

### DESCRIPTION OF EMBODIMENTS

Hereinafter, embodiments for carrying out the present invention will be described with reference to the drawings. The present invention is not limited to the embodiments described below and includes appropriately modified embodiments within a scope obvious to those skilled in the art.

Note that, in the specification of the present application, “from A to B” means “from not less than A to not more than B”.

### [1. Production Method of Fermented Milk]

A first aspect of the present invention relates to a method for producing fermented milk in which the viability of lactic acid bacteria is improved. Examples of the fermented milk produced in the present invention include yogurt. In particular, the yogurt obtained in the present invention is yogurt of pre-fermentation type in which fermentation is performed before the yogurt is packed in the container. Examples of the yogurt of pre-fermentation type include soft type yogurt, drinking type yogurt, or frozen yogurt prepared by freezing those.

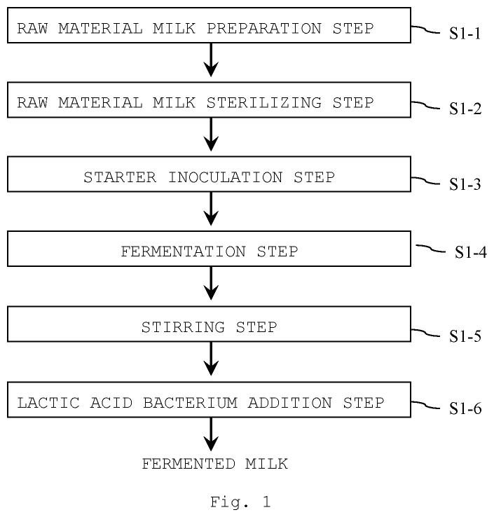

As shown in FIG. 1, the method for producing the fermented milk according to one embodiment of the present invention includes a raw material milk preparation step (S1-1), a raw material milk sterilizing step (S1-2), a starter inoculation step (S1-3), a fermentation step (S1-4), a stirring step (S1-5), and a lactic acid bacterium addition step (S1-6).

The raw material milk preparation step (S1-1) is a step of preparing raw material milk as a base material of fermented milk. The raw material milk is also called a yogurt base or a yogurt mix. The raw material milk includes one or more selected from the group consisting of milk, concentrated milk, whole fat powdered milk, skim milk, concentrated skim milk, powdered skim milk, partially-skimmed milk, partially-skimmed concentrated milk, partially-skimmed powdered milk, and a milk protein concentrate. In the present invention, publicly known raw material milk can be used. For example, the raw material milk may be composed only of raw milk (raw milk 100%). Further, the raw material milk may be prepared by mixing the raw milk with powdered skim milk, cream, water, or the like. Further, the raw material milk may be prepared by mixing (adding) sterilized milk, whole fat milk, skim milk, whole fat concentrated milk, concentrated skim milk, whole fat powdered milk, butter milk, salted butter, unsalted butter, whey, a whey powder, a whey protein concentrate (WPC), a whey protein isolate (WPI), α-La (alpha-lactalbumin), β-Lg (beta-lactoglobulin), lactose, and the like, in addition to those described above. Further, the raw material milk may be prepared by appropriately adding prewarmed gelatin, agar, a thickener, a gelling agent, a stabilizer, an emulsifier, sucrose, sweetener, fragrance, a vitamin, mineral, and the like. The raw material milk is included so that the final product has the solids not fat (SNF) content of 5% by weight or more, preferably 6% by weight or more, more preferably 8% by weight or more. The upper limit of the solids not fat content of the medium is not particularly limited. However, for example, it is preferably 30% by weight or less or 25% by weight or less. For example, the solids not fat are preferably derived from powdered skim milk. Note that about 95% of the powdered skim milk are the solids not fat and most of the remaining portion is water. Further, the medium is preferably composed of only the solids not fat and water. That is, the medium includes the solids not fat in an amount of 6% by weight or more, and the rest of the medium consists of water. Note that, needless to say, the concentration of the raw material milk can be previously adjusted to a high concentration, considering that the prepared fermented milk is added with sugar syrup and/or a preparation (fruit sauce, etc.).

Further, the raw material milk preparation step may include a treatment for homogenizing the raw material milk. In the homogenizing treatment, particles (fat globules) constituted by proteins and/or fats mainly included in the raw material milk are finely crushed (micronized). The homogenizing treatment may be performed only once, twice, or three times or more. Examples of a method for homogenizing the raw material milk include a method in which the raw material milk is passed through narrow gaps while being pushed out by pressure and a method in which the raw material milk is passed through narrow gaps while being sucked by evacuation.

The raw material milk sterilizing step (S1-2) is a step of sterilizing the raw material milk, for example, by heating. In the sterilizing step, a heating treatment needs to be performed by adjusting a heating temperature and a heating time to such an extent that various bacteria in the raw material milk can be sterilized. For example, the heating temperature of the raw material milk is preferably 80° C. or higher, particularly preferably 90° C. or higher. A publicly known method can be used for the heating treatment. For example, the heating treatment such as a high-temperature short-time (HTST) sterilizing treatment and an ultra-high temperature (UHT) sterilizing treatment may be performed. In the HTST sterilizing treatment, the raw material milk is heated at from 80° C. to 100° C. for from 3 minutes to 15 minutes. Further, in the UHT sterilizing treatment, the raw material milk is heated at from 110° C. to 150° C. for from 1 to 30 seconds. The HTST sterilizing treatment is suitable for soft type yogurt, while the UHT sterilizing treatment is suitable for drinking type yogurt.

Further, after the raw material milk is sterilized by heating, the raw material milk having a high temperature is preferably cooled to a temperature range suitable for fermentation (fermentation temperature range) before the starter inoculation step. The fermentation temperature refers to a temperature at which a microorganism (a lactic acid bacterium, etc.) becomes active and proliferation of the microorganism is promoted. For example, the fermentation temperature range of the raw material milk is generally from 30 to 60° C. In the present invention, the raw material milk having a high temperature after the heat sterilization is cooled to, for example, a culture temperature range of preferably from 30 to 60° C., more preferably from 40 to 50° C.

The starter inoculation step (S1-3) is a step of inoculating (adding) the starter to the raw material milk which is cooled to the fermentation temperature range after the heat sterilization. Note that, in the starter inoculation step, the starter may be inoculated after the raw material milk subjected to the heat sterilization is cooled to a predetermined temperature, or the starter may be inoculated while the raw material milk subjected to the heat sterilizing step is being cooled to a predetermined temperature. The starter is added to the raw material milk preferably in an amount of 0.01% by weight or more relative to the raw material milk. Specifically, the starter is added to the raw material milk in an amount of from 0.01 to 15% by weight, from 0.05 to 10% by weight, or from 0.11 to 5% by weight, relative to the raw material milk. Note that, in the specification of the present application, the raw material milk to which the starter is inoculated is also referred to as a “fermented milk base material”.

The starter includes at least one of a lactic acid bacterium or yeast. In particular, the starter preferably includes a Lactobacillus bulgaricus as the lactic acid bacterium. The “Lactobacillus bulgaricus” refers to Lactobacillus delbrueckii. subsp. bulgaricus. Further, the starter preferably includes a Streptococcus thermophilus in addition to the Lactobacillus bulgaricus. The “Streptococcus thermophilus” refers to Streptococcus thermophilus. Further, in the present invention, the starter may include a publicly known lactic acid bacterium in addition to or instead of the Lactobacillus bulgaricus and the Streptococcus thermophilus. Examples of the publicly known lactic acid bacterium include Lactobacillus gasseri, Lactobacillus plantarum, Lactobacillus casei, Lactococcus lactis, Lactococcus lactis subsp. cremoris, and Bifidobacterium.

The fermentation step (S1-4) is a step of fermenting the raw material milk using the starter. In the fermentation step, the raw material milk to which the starter is inoculated (fermented milk base material) is fermented while being kept in the fermentation temperature range (e.g., from 30 to 60° C.) to obtain fermented milk. In the present invention, in the fermentation step, a pre-fermentation treatment in which the raw material milk is fermented before being packed in the container is preferably performed. In the fermentation step, the fermentation treatment may be performed in a fermentation chamber or the like, and the fermentation treatment may also be performed in a jacket-attached tank. For example, the fermentation step may be a treatment in which the raw material milk is fermented in a fermentation chamber while a temperature (fermentation temperature) inside the fermentation chamber is maintained at about from 30° C. to 60° C. or a treatment in which the raw material milk is fermented in a jacket-attached tank while a temperature (fermentation temperature) inside the jacket-attached tank is maintained at from 30 to 60° C.

Note that, in the fermentation step, as fermentation conditions of the raw material milk, the fermentation temperature, the fermentation time, and the like are appropriately adjusted in consideration of the type and quantity of the raw material milk and the lactic acid bacteria, the flavor and texture of the fermented milk, and the like. For example, in the fermentation step, the raw material milk is preferably maintained in the fermentation temperature range for 1 hour or more. Specifically, in the fermentation step, a period of time for maintaining the raw material milk (fermentation time) is preferably from 1 hour to 12 hours, more preferably from 2 hours to 8 hours, further more preferably from 3 hours to 5 hours. Further, in the fermentation step, the fermentation conditions of the raw material milk may be appropriately adjusted so that the fermented milk after the fermentation reaches the predetermined lactic acid acidity (acidity) or pH. For example, the fermentation step is preferably continued until the lactic acid acidity of the fermented milk reaches 0.7% or more or 0.8% or more in a case where the solids not fat (SNF) content is from 8% by weight to 10% by weight. Note that the acidity (lactic acid acidity) of the raw material milk can be measured according to “Testing Methods of Compositional Standards of Milk, etc.” of Ministerial Ordinance on Milk and Milk products.

Specifically, to 10 g of a sample, 10 mL of ion exchange water free from carbon dioxide is added and then 0.5 mL of a phenolphthalein solution as an indicator is added. Then, the sample is titrated by adding a sodium hydroxide solution (0.1 mol/L) with a limit being set at the point where a pale red color does not disappear. The content of lactic acid per 100 g of the sample is calculated from the titration amount of the sodium hydroxide solution at the limit point, and this value is determined to be the acidity (lactic acid acidity). Note that the phenolphthalein solution is prepared by dissolving 1 g of phenolphthalein in an ethanol solution (50%), followed by filling up to 100 mL with the ethanol solution.

The stirring step (S1-5) is a step of stirring the fermented milk. Note that the stirring step may be performed after the fermentation step or simultaneously with the fermentation step. That is, in the former case, the fermented milk to be stirred is already fermented to the predetermined lactic acid acidity and solidified. In the latter case, the raw material milk to which the starter is inoculated is stirred while being fermented. The fermented milk in a liquid or a paste form can be obtained by performing the stirring step. The stirring speed is not particularly limited. However, the stirring is preferably performed at a relatively high speed in order to prevent the occurrence of an aggregate in the fermented milk. For example, in a case where 1,000 g of the fermented milk is stirred with a T-shaped stirring blade having a diameter of 10 cm using a stainless-steel vat having a volume of 3.0 L (diameter of 15 cm), the lower limit value of the stirring speed is preferably 30 rpm, 50 rpm, 100 rpm, 150 rpm, 160 rpm, or 200 rpm, and the upper limit value of the stirring speed is preferably 500 rpm, 400 rpm, 350 rpm, 300 rpm, or 250 rpm. The stirring treatment may be continuously performed or may be intermittently performed. However, the stirring treatment is preferably continuously performed from the standpoints of removing carbon dioxide and reducing floating of a solid matter. Note that, in the stirring step, a publicly known paddle-type stirring blade, mixer, or food cutter can be used. Further, a shearing force (N/m²) for shearing the raw material milk during the stirring step can be appropriately adjusted depending on models, operation conditions, and the like of the stirring machine (shearing machine).

The lactic acid bacterium addition step (S1-6) is a step of adding the lactic acid bacterium to the fermented milk after the stirring step. The lactic acid bacterium added to the fermented milk in this step is, unlike the lactic acid bacterium included in the starter, not for fermenting the raw material milk, but for providing a function of adjusting the body's internal environment to the fermented milk. For this reason, as the lactic acid bacterium in this step, a so-called probiotic lactic acid bacterium is adopted. The probiotic lactic acid bacterium is a lactic acid bacterium which causes a beneficial effect to a host by improving a balance of intestinal bacterial flora of the host. Examples of the probiotic lactic acid bacterium include Lactobacillus gasseri, Lactobacillus bulgaricus, or Lactobacillus casei. Examples of Lactobacillus gasseri includes a Lactobacillus gasseri OLL2716 strain and a Lactobacillus gasseri OLL2959 strain. Examples of Lactobacillus bulgaricus include a Lactobacillus delbrueckii subsp. bulgaricus OLL1073R-1 strain. Examples of Lactobacillus casei includes a Lactobacillus casei strain shirota YIT9029 strain. In the present invention, in particular, Lactobacillus gasseri is preferably adopted as the probiotic lactic acid bacterium. As the lactic acid bacterium, these probiotic lactic acid bacteria may be added either singly or as a mixture of two or more thereof. Further, as the lactic acid bacterium, frozen concentrated bacteria, a frozen pellet, a lyophilized powder, and the like can be used. Note that the lactic acid bacterium addition step (S1-6) may be performed before the stirring step (S1-5) described above.

The lactic acid bacterium used in the viability improving method of the present invention is not particularly limited. However, a Lactobacillus or a Bifidobacterium is preferable. Examples of the Lactobacillus bacterium include L. acidophilus, L. amylovorus, L. brevis, L. buchneri, L. casei, L. casei subsp. rhamnosus, L. crispatus, L. delbrueckii subsp. bulgaricus, L. delbrueckii subsp. lactis, L. fermentum, L. gallinarum, L. gasseri, L. helveticus, L. helveticus subsp. jugurti, L. johnsonii, L. kefir, L. oris, L. paracasei subsp. paracasei, L. paraplantarum, L. pentosus, L. plantarum, L. reuteri, L. salivalius, and L. zeae. Of these, L. gasseri is particularly preferable. Examples of the Bifidobacterium include B. adolescentis, B. animalis, B. bifidum, B. breve, B. catenulatum, B. globosum, B. infantis, B. lactis, B. longum, B. pseudocatenulatum, and B. suis. Of these, B. bifidum is particularly preferable. Note that these lactic acid bacteria may be used either singly or as a mixture of two or more thereof.

The lactic acid bacteria are preferably added in an amount of 0.01% by weight or more relative to the weight of the fermented milk base material. Specifically, the lactic acid bacteria are added in an amount of from 0.01 to 15% by weight, from 0.02 to 10% by weight, or from 0.03 to 5% by weight, relative to the fermented milk base material. The number of the lactic acid bacteria added to the fermented milk is preferably 10⁷ cfu/g or more, more preferably 10⁸ cfu/g or more, particularly preferably 10⁹ cfu/g or more.

The method for producing the fermented milk according to the present invention may further include a cooling step of cooling the fermented milk after the fermentation. The cooling step may be performed after the fermentation step but before the lactic acid bacterium addition step, or after the lactic acid bacterium addition step. Cooling the fermented milk reduces the progress of the fermentation. In this step, the fermented milk is cooled to a temperature lower than the fermentation temperature range (e.g., from 30 to 60° C.). For example, the fermented milk is preferably cooled to 15° C. or lower. Specifically, the fermented milk is cooled to preferably from 1 to 15° C., more preferably from 3 to 12° C., further more preferably from 5 to 10° C. Cooling the fermented milk to a temperature suitable for food in this manner can reduce or prevent a change in the flavor (sourness) and the texture (mouthfeel), and physical properties (hardness, etc.) of the fermented milk.

According to the present invention, it becomes possible to improve the viability of the lactic acid bacteria added in the above lactic acid bacterium addition step during storage period of the fermented milk. That is, it becomes possible to moderate the reduction of the lactic acid bacteria during storage period of the fermented milk. Specifically, in a state where the lactic acid bacteria are added to the fermented milk and the added fermented milk is stored at 5° C., when the number of the lactic acid bacteria on storage period day 1 (one day after addition of the lactic acid bacteria) is taken as 100%, the survival rate of the lactic acid bacteria on storage period day 16 (16 days after addition of the lactic acid bacteria) is preferably 50% or more, more preferably 60% or 70% or more, particularly preferably 80% or more. Further, the survival rate of the lactic acid bacteria on storage period day 25 (16 days after addition of the lactic acid bacteria) under the same conditions is preferably 30% or more, more preferably 40% or 50% or more, particularly preferably 60% or more.

Conventionally, the probiotic lactic acid bacteria are usually added to the raw material milk together with the starter before fermentation. In this case, the survival rate of the probiotic lactic acid bacteria on storage period day 16 under the above conditions is lower than 50%, and, after the lapse of 25 days of the storage period, the survival rate decreases to about 10% or less. In contrast, as described in the present invention, when the probiotic lactic acid bacteria are added to the fermented milk after fermentation, the survival rate of the probiotic lactic acid bacteria can be dramatically improved as described above. Thus, the present invention can contribute to stabilizing the quality of the fermented milk product and extending the expiration date thereof. Further, the improvement of the viability of the lactic acid bacteria can reduce the addition amount of the lactic acid bacteria, thus making it possible to reduce the raw material cost.

### [2. Production Method of Lactic Acid Bacterium Starter]

A second aspect of the present invention relates to a method for producing a lactic acid bacterium starter used for producing the fermented milk. In particular, according to the present invention, the viability of the lactic acid bacterium starter can be improved.

The method for producing the lactic acid bacterium starter is a method for producing the lactic acid bacterium starter used for fermenting the raw material milk by culturing a lactic acid bacterium as a seed bacterium in a medium and performing intermediate fermentation. The “lactic acid bacterium starter” includes a substance obtained by culturing a lactic acid bacterium in a medium (solution) and performing intermediate fermentation. The lactic acid bacterium starter basically includes the lactic acid bacterium and the medium solution in which the lactic acid bacterium is cultured as constituent elements. Further, the lactic acid bacterium starter includes not only the one directly inoculated to the raw material milk serving as a base material of the fermented milk, but also the one prepared by inoculating such a lactic acid bacterium starter to another medium and further propagating (scaling up) the lactic acid bacteria over the following generations.

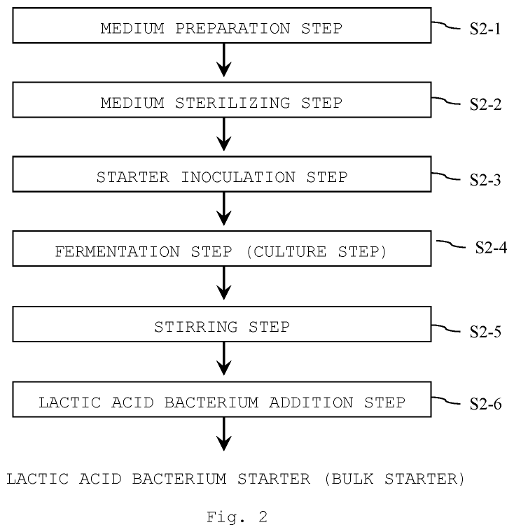

As shown in FIG. 2, the method for producing the lactic acid bacterium starter according to one embodiment of the present invention includes a medium preparation step (S2-1), a medium sterilizing step (S2-2), a starter inoculation step (S2-3), a fermentation step (culture step) (S2-4), a stirring step (S2-5), and a lactic acid bacterium addition step (S2-6).

The medium preparation step (S2-1) is a step of preparing a medium to which the lactic acid bacterium is inoculated. The medium is a solution for culturing the lactic acid bacterium. The number of the lactic acid bacterium can be increased by inoculating the lactic acid bacterium to the medium and culturing the lactic acid bacterium in the medium. The medium has the solids not fat (SNF) content of 6% by weight or more, preferably 8% by weight or more, more preferably 9% by weight or more. The upper limit of the solids not fat content of the medium is not particularly limited. However, for example, it is preferably 30% by weight or less or 25% by weight or less. In particular, the solids not fat are preferably derived from powdered skim milk. Note that about 95% of the powdered skim milk are the solids not fat and most of the remaining portion is water. Further, the medium is preferably composed of only the solids not fat and water. That is, the medium includes the solids not fat in an amount of 6% by weight or more, and the rest of the medium consists of water.

The medium sterilizing step (S2-2) is a step of sterilizing the medium prepared in the medium preparation step, for example, by heating. In the sterilizing step, a heating treatment needs to be performed by adjusting a heating temperature and a heating time to such an extent that various bacteria in the medium can be sterilized. In the present invention, the medium is preferably heated to 80° C. or higher, 90° C. or higher, 95° C. or higher, or 100° C. or higher. A publicly known method can be used for the heat sterilization. For example, for the heat sterilization, the heating treatment may be performed using a plate-type heat exchanger, a tube-type heat exchanger, a steam injection-type heating apparatus, a steam infusion-type heating apparatus, a conduction-type heating apparatus, and the like.

The heating treatment may be performed using a jacket-attached tank. Note that the sterilization of the medium is not limited to the heating, and, for example, a publicly known method such as ultraviolet ray irradiation can be employed.

Further, in a case where the medium is subjected to the sterilization treatment by heating, the medium having a high temperature is preferably cooled to a temperature range suitable for culturing the lactic acid bacterium (culture temperature range) before the lactic acid bacterium addition step. The culture temperature range refers to a temperature at which a microorganism (a lactic acid bacterium, etc.) becomes active and proliferation of the microorganism is promoted. For example, the culture temperature range of the lactic acid bacterium is generally from 30 to 60° C. In the present invention, the medium having a high temperature after the heat sterilization is cooled to, for example, the culture temperature range of preferably from 30 to 60° C., more preferably from 35 to 55° C.

The starter inoculation step (S2-3) is a step of inoculating (adding) the starter to the medium in the culture temperature range. Note that, in the starter inoculation step, the starter may be inoculated after the medium subjected to the heat sterilization is cooled to a predetermined temperature, or the lactic acid bacterium may be inoculated while the medium subjected to the heat sterilization is being cooled to a predetermined temperature. In the starter inoculation step, the starter is preferably added to the medium in an amount of 0.05% by weight or more relative to the medium. Specifically, the starter is added to the medium in an amount of from 0.05 to 10% by weight or from 0.1 to 5% by weight, relative to the medium. Further, as the starter, the same starter as described in the starter inoculation step (S1-3) in the production method of the fermented milk can be adopted.

The fermentation step (culture step) (S2-4) is a step of culturing the lactic acid bacterium included in the starter in the medium and propagating the lactic acid bacterium. Culturing of the lactic acid bacterium is preferably terminated using the acidity of the medium as an indicator. The upper limit of the culture time of the lactic acid bacterium is not particularly limited. However, for example, culturing is terminated when fermentation of the medium proceeds and the acidity of the medium reaches a predetermined value. In this step, for example, the acidity for terminating the culturing is preferably set to 0.6%, 0.7%, 0.75%, or 0.8%. It is only required to be set to a range of from 0.6 to 1.2%. Note that the acidity (lactic acid acidity) of the medium is measured according to “Testing Methods of Compositional Standards of Milk, etc.” of Ministerial Ordinance on Milk and Milk products.

The stirring step (S2-5) is a step of stirring the medium. Note that the stirring step may be performed after the fermentation step or simultaneously with the fermentation step. Further, stirring conditions are the same as described in the stirring step (S1-5) in the production method of the fermented milk.

The lactic acid bacterium addition step (S2-6) is a step of adding the lactic acid bacterium to the medium after stirring. As the lactic acid bacterium used in this step, a so-called probiotic lactic acid bacterium is adopted. Examples of the probiotic lactic acid bacterium include Lactobacillus gasseri, Lactobacillus bulgaricus, or Lactobacillus casei. Examples of Lactobacillus gasseri include a Lactobacillus gasseri OLL2716 strain and a Lactobacillus gasseri OLL2959 strain. Examples of Lactobacillus bulgaricus include a Lactobacillus delbrueckii subsp. bulgaricus OLL1073R-1 strain. Examples of Lactobacillus casei include a Lactobacillus casei strain shirota YIT902 9 strain. In the present invention, in particular, Lactobacillus gasseri is preferably adopted as the probiotic lactic acid bacterium. As the lactic acid bacterium, these probiotic lactic acid bacteria may be added either singly or as a mixture of two or more thereof. Further, as the lactic acid bacterium, frozen concentrated bacteria, a frozen pellet, a lyophilized powder, and the like can be used. Note that the lactic acid bacterium used in the viability improving method of the present invention is not particularly limited. However, a Lactobacillus bacterium or a Bifidobacterium is preferable. Examples of the Lactobacillus bacterium and the Bifidobacterium are as described above. Further, the lactic acid bacterium addition step (S2-6) may be performed before the stirring step (S2-5) described above.

The method for producing the lactic acid bacterium starter according to the present invention may further include a cooling step of cooling the medium after the culturing. The cooling step may be performed after the fermentation step but before the lactic acid bacterium addition step, or after the lactic acid bacterium addition step. In this step, the medium is cooled to a temperature lower than the fermentation temperature range (e.g., from 30 to 60° C.). For example, the medium is preferably cooled to 15° C. or lower. Specifically, the medium is cooled to preferably from 1 to 15° C., more preferably from 3 to 12° C., further more preferably from 5 to 10° C.

According to the present invention, it becomes possible to improve the viability of the lactic acid bacteria added in the above lactic acid bacterium addition step during a storage period. Specifically, in a state where the lactic acid bacteria are added to the medium and the resulting medium is stored at 5° C., when the number of the lactic acid bacteria on storage period day 1 (one day after addition of the lactic acid bacteria) is taken as 100%, the survival rate of the lactic acid bacteria on storage period day 16 (16 days after addition of the lactic acid bacteria) is preferably 50% or more, more preferably 60% or 70% or more, particularly preferably 80% or more or 90% or more. Further, similarly, the survival rate of the lactic acid bacteria on storage period day 25 (16 days after addition of the lactic acid bacteria) under the same conditions is preferably 50% or more, more preferably 60% or 70% or more, particularly preferably 80% or more or 90% or more. As described above, according to the present invention, the viability of the lactic acid bacteria during the storage period can be improved, thus making it possible to obtain the lactic acid bacterium starter suitable for the long-period storage.

### EXAMPLES

### Test Example 1: Drinkable Yogurt

### Example 1

Using a stainless-steel vat having a volume of 3.0 L (diameter of 15 cm), 83.5 g of powdered skim milk, 10.0 g of fresh cream, and 488.3 g of tap water were mixed to prepare a base mix (raw material milk). This base mix was subjected to heat sterilization at 95° C. for 5 minutes and then cooled to 43° C. Subsequently, this base mix was inoculated with 18.0 g (3% by weight of the fermented milk base total) of the starter and fermented at 43° C. As the starter, bacteria including a Lactobacillus bulgaricus and a Streptococcus thermophilus, which were isolated from a trade name “Meiji Bulgaria Yogurt Zero Fat Fresh Strawberries” (Meiji Co., Ltd.) and cultured in a 10% powdered skim milk medium, were used. The fermentation was terminated at the time point when the lactic acid acidity of the base mix (fermented milk base material) to which the starter was inoculated reached 1.2% (pH=4.5), and the curd thus obtained was cooled to 5° C. while being crushed at 350 rpm using a T-shaped stirring blade having a diameter of 10 cm in a state where the vat was dipped in ice water. Subsequently, to the base mix, 0.24 g (0.04% by weight of the fermented milk base total) of frozen concentrated bacteria of Lactobacillus gasseri OLL2716 Lactobacillus gasseri OLL2716 (accession number: FERM BP-6999) were added. Subsequently, a homogenizing treatment was aseptically performed at 15 MPa for turning the base mix into a liquid form, thereby producing a fermented milk base (fermented milk) (a total of 600.0 g).

Using a stainless-steel vat having a volume of 1.0 L, 92.0 g of glucose-fructose syrup, 4.7 g of sugar, 2.5 gm of pectin, and 300.8 g of tap water were mixed, and the resulting mixture was heated (sterilized) at 95° C. for 1 minute and then cooled to 5° C. to produce sugar syrup (a total of 400.0 g).

The above fermented milk base in an amount of 600.0 g and the above sugar syrup in an amount of 400.0 g were mixed to prepare drinkable yogurt of Example 1, and the drinkable yogurt was dispensed in a PET container (a capacity of 100 g) and stored in a refrigerator at 5° C.

The drinkable yogurt of Example 1 produced by the above method was stored at 5° C., and the number of the Lactobacillus gasseri OLL2716Lactobacillus gasseri OLL2716 was measured on storage day 1, day 8, day 16, and day 25.

As shown in Table 1 below, the Lactobacillus gasseri OLL2716Lactobacillus gasseri OLL2716 survival rate of the drinkable yogurt according to Example 1 during storage was 86.1% on day 8, 85.0% on day 16, and 65.0% on day 25, when the number of the Lactobacillus gasseri OLL2716 on storage day 1, 9.0×10⁷ cfu/g, was taken as 100%.

### Comparative Example 1

Under the same conditions as in Example 1, the base mix (fermented milk) was prepared and subjected to the heat sterilization and cooling. Subsequently, to this base mix, 18.0 g (3% by weight of the fermented milk base total) of the same starter as in Example 1, as well as 0.24 g (0.04% by weight of the fermented milk base total) of frozen concentrated bacteria of “Lactobacillus gasseri OLL2716” (Lactobacillus gasseri OLL2716) (accession number: FERM BP-6999), were inoculated, followed by fermentation at 43° C. The fermentation was terminated at the time point when the lactic acid acidity of the base mix (fermented milk base material) to which the starter and the Lactobacillus gasseri OLL2716 were inoculated reached 1.2% (pH=4.5), and the curd thus obtained was cooled to 5° C. while being crushed at 350 rpm using a T-shaped stirring blade having a diameter of 10 cm in a state where the vat was dipped in ice water. Subsequently, a homogenizing treatment was aseptically performed at 15 MPa for turning the base mix into a liquid form, thereby producing a fermented milk base (fermented milk) (a total of 600.0 g). Further, the sugar syrup in an amount of 400.0 g was produced under the same conditions as in Example 2 and mixed with 600.0 g of the above fermented milk base to produce drinkable yogurt of Comparative example 1. The drinkable yogurt was dispensed in a PET container (a capacity of 100 g) and stored in a refrigerator at 5° C.

The drinkable yogurt of Comparative example 1 produced by the above method was stored at 5° C., and the number of the Lactobacillus gasseri OLL2716 was measured on storage day 1, day 8, day 16, and day 25. The number of the lactic acid bacteria was measured by the same method as in Example 1.

As shown in Table 1 below, the Lactobacillus gasseri OLL2716 survival rate of the drinkable yogurt according to comparative example 1 during storage was 34.7% on day 8, 39.1% on day 16, and 6.2% on day 25, when the number of the Lactobacillus gasseri OLL2716 on storage day 1, 13.6×10⁷, was taken as 100%.

TABLE 1
Text example 1: Drinkable yogurt
Lactobacillus gasseri OLL2716 survival rate

| | Day 1 | Day 8 | Day 16 | Day 25 |
|---|---|---|---|---|
| Example 1 | 100% | 86.1% | 85.0% | 65.0% |
| | (9.0 × 10 ^{∧} 7 cfu/g) | | | |
| Comparative | 100% | 34.7% | 39.1% | 6.2% |
| example 1 | (13.6 × 10 ^{∧} 7 cfu/g) | | | |

### [Discussion]

As shown in Table 1 above, in the drinkable yogurt of Example 1, 85% of the Lactobacillus gasseri OLL2716 survived after the lapse of 16 days which is commonly applied as expiration date, while, in the drinkable yogurt of Comparative example 1, the number of the Lactobacillus gasseri OLL2716 decreased to about 40% or less at the same time point. Thus, in the drinkable yogurt of Example 1, the survival rate of the Lactobacillus gasseri OLL2716 is twice or more than that of Comparative example 1 after the lapse of 16 days, confirming that the survival rate significantly increased. Further, after the lapse of 25 days, the Lactobacillus gasseri OLL2716 rarely survived in Comparative example 1, while, 65% of the Lactobacillus gasseri OLL2716 survived in Example 1. Thus, according to the present invention, an effect of extending the expiration date of the drinkable yogurt can be expected. Further, in Example 1, the Lactobacillus gasseri OLL2716 are added after the base mix is fermented in order to improve the viability of this bacteria strain. However, it is speculated that the effect of improving the viability is not limited to the Lactobacillus gasseri OLL2716 and applied to the same species, Lactobacillus gasseri, or other lactic acid bacteria.

### Test Example 2: Soft Yogurt

### Example 2

Using a stainless-steel vat having a volume of 3.0 L (diameter of 15 cm), 781.0 g of raw milk, 27.4 g of powdered skim milk, 60.0 g of sugar, and 101.4 g of tap water were mixed to prepare a base mix (raw material milk). This base mix was heated (sterilized) at 95° C. for 5 minutes and then cooled to 43° C. Subsequently, the base mix was inoculated with 30.0 g (3% by weight of the fermented milk total) of the same starter as in Example 1 and then fermented at 43° C. The fermentation was terminated at the time point when the lactic acid acidity of the base mix (fermented milk base material) to which the starter was inoculated reached 0.7% (pH=4.5), and the curd thus obtained was passed through a 60 mesh filter to perform a smoothing treatment. Subsequently, the treated curd was cooled to 5° C. while being stirred at 300 rpm for 30 minutes using a T-shaped stirring blade having a diameter of 10 cm in a state where the vat was dipped in ice water. Subsequently, to the base mix, 0.24 g (0.024% by weight of the fermented milk total) of frozen concentrated bacteria of “Lactobacillus gasseri OLL2716” (Lactobacillus gasseri OLL2716) (accession number: FERM BP-6999) were added to produce soft yogurt (fermented milk) of Example 2 (a total of 1,000 g).

The soft yogurt of Example 2 produced by the above method was stored at 5° C., and the number of the Lactobacillus gasseri OLL2716 was measured on storage day 1, day 16, and day 25. The number of the lactic acid bacteria was measured by the same method as in Example 1. As shown in Table 2 below, the Lactobacillus gasseri OLL2716 survival rate of the soft yogurt according to Example 2 during storage was 82.7% on day 16 and 74.7% on day 25, when the number of the Lactobacillus gasseri OLL2716 on storage day 1, 4.0×10⁷ cfu/g, was taken as 100%.

### Comparative Example 2

Under the same conditions as in Example 2, the base mix (fermented milk) was prepared and subjected to the heat sterilization and cooling. To this base mix, 18.0 g (3% by weight of the fermented milk total) of the same starter as in Example 1, as well as 0.24 g (0.024% by weight of the fermented milk total) of frozen concentrated bacteria of “Lactobacillus gasseri OLL2716” (accession number: FERM BP-6999), were inoculated, followed by fermentation at 43° C. The fermentation was terminated at the time point when the lactic acid acidity of the base mix (fermented milk base material) to which the starter and the Lactobacillus gasseri OLL2716 were inoculated reached 0.7% (pH=4.5), and the curd thus obtained was passed through a 60 mesh filter to perform a smoothing treatment. The treated curd was cooled to 5° C. while being stirred at 300 rpm for 30 minutes using a T-shaped stirring blade having a diameter of 10 cm in a state where the vat was dipped in ice water. Subsequently, the curd was cooled to 5° C. while being stirred at 300 rpm for 30 minutes in a state where the vat was dipped in ice water to produce soft yogurt (fermented milk) of Comparative example 2 (a total of 1,000 g).

The soft yogurt of Comparative example 2 produced by the above method was stored at 5° C., and the number of the Lactobacillus gasseri OLL2716 was measured on storage day 1, day 16, and day 25. The number of the lactic acid bacteria was measured by the same method as in Example 1. As shown in Table 2 below, the Lactobacillus gasseri OLL2716 survival rate of the soft yogurt according to Example 2 during storage was 25.5% on day 16 and 10.9% on day 25, when the number of the Lactobacillus gasseri OLL2716 on storage day 1, 4.0×10⁷ cfu/g, was taken as 100%.

TABLE 2
Test example 2: Soft yogurt
Lactobacillus gasseri OLL2716 survival rate

| | Day 1 | Day 16 | Day 25 |
|---|---|---|---|
| Example 2 | 100% | 82.7% | 74.7% |
| | (4.0 × 10 ^{∧} 7 cfu/g) | | |
| Comparative | 100% | 25.5% | 10.9% |
| example 2 | (4.0 × 10 ^{∧} 7 cfu/g) | | |

### DISCUSSION

As shown in Table 2 above, in the soft yogurt of Example 2, 82.7% of the Lactobacillus gasseri OLL2716 survived after the lapse of 16 days which is commonly applied as expiration date, while, in the soft yogurt of Comparative example 1, the number of the Lactobacillus gasseri OLL2716 decreased to about 25% at the same time point. Thus, in the soft yogurt of Example 2, the survival rate of the Lactobacillus gasseri OLL2716 was three times or more than that of Comparative example 2 after the lapse of 16 days, confirming that the survival rate significantly increased. Further, after the lapse of 25 days, only about 10% of the Lactobacillus gasseri OLL2716 survived in Comparative example 2, while, 74.7% of the Lactobacillus gasseri OLL2716 survived in Example 2. Thus, according to the present invention, the effect of extending the expiration date of the soft yogurt can be also expected. It is speculated that the effect of improving the viability is not limited to the Lactobacillus gasseri OLL2716 and applied to the same species, Lactobacillus gasseri, or other lactic acid bacteria.

### Test Example 3: Bulk Starter

### Example 3

Using a stainless-steel vat having a volume of 3.0 L (diameter of 15 cm), 100 g of powdered skim milk and 870 g of tap water were mixed to prepare a bulk starter base (medium). This bulk starter base was heated (sterilized) at 95° C. for 5 minutes and then cooled to 40° C. Next, the bulk starter base was inoculated with 30 g (3% by weight) of the same starter as in Example 1. Subsequently, the bulk starter base was subjected to static fermentation at 40° C. until the lactic acid acidity reached 0.70%, and then the curd was cooled to 25° C. while being stirred at 350 rpm using a T-shaped stirring blade having a diameter of 10 cm with the container dipped in cold water. The curd was maintained at this temperature for 2 hours and then cooled to 5° C. To the curd, 13.2 g (1.32% by weight of the bulk starter base total) of frozen concentrated bacteria of “Lactobacillus gasseri OLL2716” (Lactobacillus gasseri OLL2716) (accession number: FERM BP-6999) were added to produce a bulk starter of Example 3.

The bulk starter of Example 3 produced by the above method was stored at 5° C., and the number of the Lactobacillus gasseri OLL2716 was measured on storage day 1, day 8, day 16, and day 25.

As shown in Table 3 below, the Lactobacillus gasseri OLL2716 survival rate of the bulk starter according to Example 3 during storage was 98% on day 25, when the number of the Lactobacillus gasseri OLL2716 on storage day 1, 9.2×10⁸ cfu/g, was taken as 100%.

### Comparative Example 3

Under the same conditions as in Example 3, the bulk starter base (medium) was prepared and subjected to the heat sterilization and cooling. To this bulk starter base, 30.0 g (3% by weight) of the same starter as in Example 1, as well as 13.2 g (1.32% by weight of the bulk starter base total) of frozen concentrated bacteria of “Lactobacillus gasseri OLL2716” (Lactobacillus gasseri OLL2716) (accession number: FERM BP-6999), were inoculated. Subsequently, the bulk starter base was subjected to static fermentation at 40° C. until the lactic acid acidity reached 0.70%, and then the curd was cooled to 25° C. while being stirred at 350 rpm using a T-shaped stirring blade having a diameter of 10 cm with the container dipped in cold water. The curd was maintained at this temperature for 2 hours and then cooled to 5° C. to produce the bulk starter of Comparative example 3.

The bulk starter of Comparative example 3 produced by the above method was stored at 5° C., and the number of the Lactobacillus gasseri OLL2716 was measured on storage day 1 and day 25. The number of the lactic acid bacteria was measured as described above. As shown in Table 3 below, the Lactobacillus gasseri OLL2716 survival rate of the bulk starter according to Comparative example 3 during storage was 0% on day 25, when the number of the Lactobacillus gasseri OLL2716 on storage day 1, 13.3×10⁸ cfu/g, was taken as 100%.

TABLE 3
Test example 3: Bulk starter
Lactobacillus gasseri OLL2716 survival rate

| | Day 1 | Day 25 |
|---|---|---|
| Example 3 | 100% | 98% |
| | (9.2 × 10 ^{∧} ⁸ cfu/g) | |
| Comparative | 100% | 0% |
| example 3 | (13.3 × 10 ^{∧} ⁸ cfu/g) | |

### [Discussion]

As shown in Table 3 above, in the bulk starter of Example 3, almost 100% of the Lactobacillus gasseri OLL2716 survived after the lapse of 16 days and 25 days, while, in the bulk starter of Comparative example 3, most of the Lactobacillus gasseri OLL2716 died out after the lapse of 25 days. Thus, according to the present invention, it was found that the storage period of the bulk starter could be dramatically extended. It is speculated that the effect of improving the viability is not limited to the Lactobacillus gasseri OLL2716 and applied to the same species, Lactobacillus gasseri, or other lactic acid bacteria.

Note that depositary information on the “Lactobacillus gasseri OLL2716” (Lactobacillus gasseri OLL2716 strain) used in the above Test examples is as follows.

(1) Name of depositary institute: International Patent Organism Depositary, National Institute of Advanced Industrial Science and Technology

(2) Contact information: Central 6, 1-1, Higashi 1-chome, Tsukuba-shi, Ibaraki-ken, zip code: 305-8566

(3) Accession number: FERM BP-6999

(4) Identification reference: Lactobacillus gasseri OLL2716

(5) Date of original deposit: May 24, 1999

(6) Date of transfer to deposit under the Budapest treaty: Jan. 14, 2000

Further, depositary information on the Lactobacillus gasseri OLL2959 strain described in the specification is as follows.

(1) Name of depositary institute: International Patent Organism Depositary, National Institute of Technology and Evaluation

(2) Contact information: 2-5-8 Kazusakamatari, Kisarazu-shi, Chiba-ken, zip code: 292-0818

(3) Accession number: NITE BP-224

(4) Identification reference: Lactobacillus gasseri OLL2959

(5) Date of original deposit: Mar. 31, 2006

(6) Date of transfer to deposit under the Budapest treaty: Nov. 21, 2007

In the above specification of the present application, the embodiments and examples of the present invention have been described to express the content of the present invention. However, the present invention is not limited to the above embodiments and examples, and encompasses obvious modifications and improvements made by those skilled in the art based on the matters described in the specification of the present application.

### INDUSTRIAL APPLICABILITY

The present invention relates to the method for producing the fermented milk and the method for producing the lactic acid bacterium starter, thus the present invention can be suitably used in manufacturing industries of the fermented milk such as yogurt.

1. A method for producing fermented milk comprising:
a starter inoculation step of inoculating a starter to raw material milk to obtain a fermented milk base material;
a fermentation step of fermenting the fermented milk base material to obtain fermented milk; and
a lactic acid bacterium addition step of adding a lactic acid bacterium to the fermented milk.

2. The method for producing fermented milk according to claim 1, wherein the lactic acid bacterium includes a probiotic lactic acid bacterium.

3. The method for producing fermented milk according to claim 1, wherein the lactic acid bacterium is Lactobacillus gasseri, Lactobacillus bulgaricus, Lactobacillus plantarum, Lactobacillus casei, or Bifidobacterium.

4. The method for producing fermented milk according to claim 3, wherein the starter includes both or either of Lactobacillus bulgaricus and Streptococcus thermophilus.

5. The method for producing fermented milk according to claim 1, wherein the fermentation step is a step of fermenting the fermented milk base material until an acidity of the fermented milk base material reaches 0.70 or more.

6. The method for producing fermented milk according to claim 1, further comprising a stirring step of stirring the fermented milk after the fermentation step but before the lactic acid bacterium addition step.

7. A method for producing a lactic acid bacterium starter used for fermenting raw material milk and obtaining fermented milk, the method comprising:
a starter inoculation step of inoculating a starter to a medium including a milk component;
a fermentation step of fermenting the medium after the starter inoculation step; and
a lactic acid bacterium addition step of adding a lactic acid bacterium to the medium after the fermentation step.
